(19) United States
(12) Patent Application Publication
(10) Pub. No.: US 2020/0164219 A1
(43) Pub. Date: May 28, 2020

(54) PROBE UNIT AND ACOUSTIC WAVE RECEIVING SYSTEM

(71) Applicant: CANON KABUSHIKI KAISHA, Tokyo (JP)

(72) Inventors: Masaki Hosoda, Kawasaki-shi (JP); Yohei Hashizume, Tokyo (JP); Nobuhiro Ito, Yamato-shi (JP); Kazuhiko Fukutani, Yokohama-shi (JP)

(21) Appl. No.: 16/687,133

(22) Filed: Nov. 18, 2019

(30) Foreign Application Priority Data
Nov. 22, 2018 (JP) 2018-219742

(51) Int. Cl. A61N 5/06; A61B 5/00

(52) U.S. Cl. CPC A61N 5/0616; A61B 5/0095; A61N 2005/0628; A61N 2005/0659; A61N 2005/0627; A61N 2005/0662

(57) ABSTRACT

A probe unit for receiving an acoustic wave includes a probe, an output unit, and a connector, wherein the output unit is configured to output an acoustic wave signal detected by the probe to an acoustic wave device, and the connector is configured to be detachably connected with a light irradiation unit that includes a light emitting unit and a target guide, such that the probe and the connector are disposed so as to allow an operator to visually check a region to be irradiated when the operator places the probe so as to be acoustically coupled with an object.

### BACKGROUND

### Field of the Disclosure

The present disclosure relates to a probe unit that receives an acoustic wave propagating from an object and to an acoustic wave receiving system that includes a probe unit.

### Description of the Related Art

A laser light irradiation device used in dermatology, aesthetic dermatology, and so on for providing a treatment by heating a specific region of an object is available. U.S. Patent Application Publication No. 2008/0172114 discloses a handheld laser probe including a light emitting unit that emits laser light to remedy freckles and so on. The laser probe described in U.S. Patent Application Publication No. 2008/0172114 includes a handpiece that includes the light emitting unit and a target guide that extends from the handpiece toward an object and provides a guide as to the distance between the light emitting unit and the object and as to an irradiation spot.

Further, a technique for acoustically monitoring the effect of treatment laser light on an object by using a photoacoustic signal to grasp the effect of treatment is available. U.S. Patent Application Publication No. 2008/0071172 describes a handheld probe that includes a handpiece provided with a probe and an optical fiber end from which treatment laser light is emitted. U.S. Patent Application Publication No. 2008/0071172 further discloses that changes in the monitored photoacoustic signal reflect the effect of treatment.

The region to be irradiated is usually set on the basis of a therapy plan determined before treatment or set by the doctor in accordance with the state of the object under treatment In dermatology, oral surgery, dentistry, digestive surgery, and so on, laser light irradiation is performed a plurality of times while the irradiation spot is moved discretely or continuously to cover the region to be irradiated having a size of several millimeters to several centimeters. The operator performs an operation of moving the handpiece apart from the object in each of the plurality of times of laser light irradiation in order to visually check the state after irradiation and to search for the next irradiation region. Due to an unstable procedure caused by body motion of the operator or the subject, fatigue of the operator, and so on, positional relationships including the distance and angle between the handpiece and the object might not be constant in each of the plurality of times of laser light irradiation.

In a case where the method described in U.S. Patent Application Publication No. 2008/0071172 is used to acoustically monitor the state of the irradiation region each time laser light irradiation is performed, the acoustic coupling might not be constant in each irradiation event because of the handpiece moved up and down. In a case where the method described in U.S. Patent Application Publication No. 2008/0071172 is used and the handpiece is kept in close contact with the object each time laser light irradiation is performed in order to maintain the acoustic coupling, the precision of alignment of the irradiation region may be compromised. When the acoustic coupling and the alignment are checked for each irradiation event, the procedure becomes complicated, and the time the operator takes to perform the procedure becomes longer. Accordingly, improvement in usability is desired for the operator and the subject.

### SUMMARY

The present disclosure provides a probe unit that allows acoustic monitoring of information from a treatment target without obstructing visual check of the region to be irradiated of the treatment target.

A probe unit according to an aspect of the present disclosure is a probe unit for receiving an acoustic wave, the probe unit including a probe, an output unit, and a connector, The output unit is configured to output an acoustic wave signal detected by the probe to an acoustic wave device. The connector is configured to be detachably connected with a light irradiation unit that includes a light emitting unit and a target guide. The probe and the connector are disposed so as to allow an operator to visually check a region to be irradiated when the operator places the probe so as to be acoustically coupled with an object.

Further features of the present disclosure will become apparent from the following description of exemplary embodiments with reference to the attached drawings.

### BRIEF DESCRIPTION OF THE DRAWINGS

FIG. 1A and FIG. 1B are schematic diagrams respectively illustrating a probe unit and a light irradiation unit according to one or more embodiments of the subject disclosure.

FIG. 1C to FIG. 1E are schematic diagrams illustrating a state where the probe unit and the light irradiation unit are connected with each other according to one or more embodiments of the subject disclosure.

FIG. 2 is a schematic diagram illustrating an irradiation light monitoring system according to one or more embodiments of the subject disclosure.

FIG. 3 is a schematic diagram illustrating the probe unit according to one or more embodiments of the subject disclosure.

FIG. 4A and FIG. 4B are schematic diagrams illustrating the probe unit according to one or more embodiments of the subject disclosure.

FIG. 5A and FIG. 5B are schematic diagrams illustrating the probe unit according to one or more embodiments of the subject disclosure.

FIG. 6A and FIG. 6B are schematic diagrams illustrating the probe unit according to one or more embodiments of the subject disclosure.

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DESCRIPTION OF THE EMBODIMENTS

Hereinafter, embodiments of the present disclosure will be described in detail with reference to the drawings. Note that the same constructional elements are basically given the same reference numerals, and duplicated descriptions thereof are omitted.

### First Embodiment

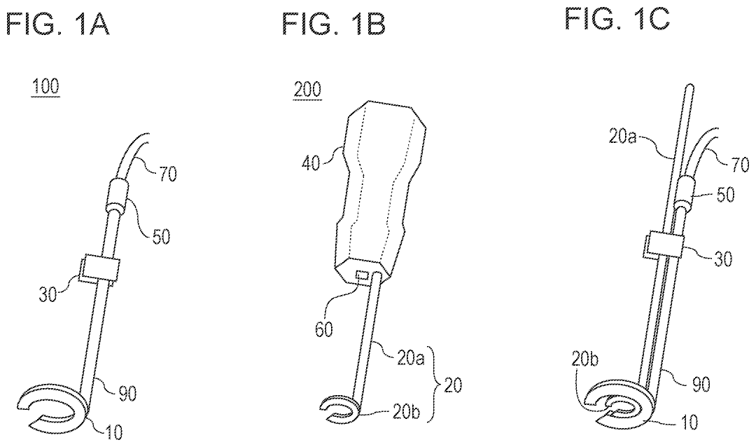

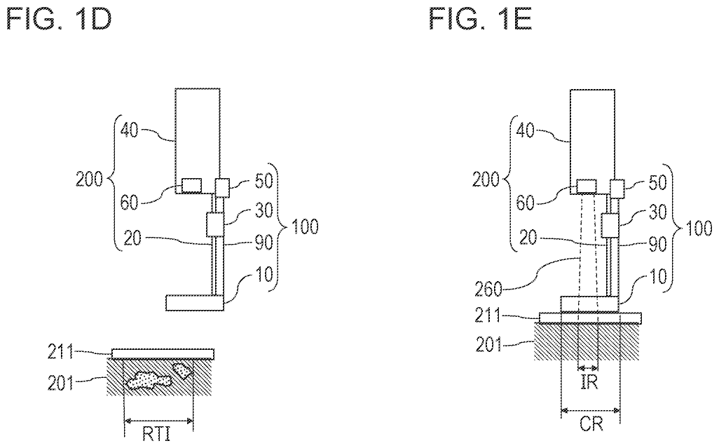

FIG. 1A and FIG. 1B are schematic diagrams respectively illustrating a probe unit 100 and a light irradiation unit 200 with which the probe unit 100 is connected according to a first embodiment, and FIG. 1C to FIG. 1E are schematic diagrams illustrating a state where the probe unit 100 and the light irradiation unit 200 are connected with each other.

As illustrated in FIG. 1A, the probe unit 100 includes a probe 10 that receives an acoustic wave from an object, an output unit 50 that outputs an acoustic wave signal detected by the probe 10 to an acoustic wave device, and a connector 30 that is detachably connected with the light irradiation unit 200 described below.

The light irradiation unit 200 is a target element with which the probe unit 100 is connected. As illustrated in FIG. 1B, the light irradiation unit 200 includes a light emitting unit 60 that radiates light toward an object and a target guide 20.

The light emitting unit 60 is disposed at a handpiece 40 that is held by the operator. The target guide 20 includes a distance guide 20a having a rod shape and extending from the handpiece 40 in the radiation direction and an irradiation region guide 20b having an arc shape, disposed at the distal end of the distance guide 20a, and provided so as to surround an irradiation spot. The target guide 20 is disposed so as not to overlap with the light emitting unit 60 on the object-side surface of the handpiece 40. In other words, the operator includes “a practitioner”, “a technician” and “a dermatologist”.

The target guide 20 is provided in the light irradiation unit 200 for the purpose of providing guides so that the luminous intensity of irradiation light on an object 201 does not fluctuate to a large degree and so that an irradiation region IR is aligned with a region to be irradiated RTI of the object 201. From the light emitting unit 60, collimated light may be radiated, or diffused light or convergent light may be radiated. Accordingly, the target guide 20 has a function of providing the operator with a guide as to a source-to-object distance (SOD), which is the distance between the light emitting unit 60 and the object 201, so that the luminous intensity that is inversely proportional to the distance SOD is less affected by a procedure performed by the operator.

The distance guide 20a provides the operator with a guide as to the distance SOD between the light emitting unit 60 and the object 201. The distance guide 20a and the irradiation region guide 20b may be integrated into one unit or may be formed of separate members, Alternatively, the target guide 20 need not include the irradiation region guide 20b as a constructional component thereof and may be formed of only the distance guide 20a.

To provide a guide as to the irradiation region IR on the object 201, the light irradiation unit 200 may be provided with a mechanism for radiating guide light that has an intensity lower than that of a light beam 260 for treatment and that includes a visible light component. Unlike the light beam 260 for treatment, the guide light is set so as to be minimally invasive. The guide light may be radiated simultaneously with main irradiation in which the light beam 260 for treatment is radiated, before main irradiation, or after main irradiation. The region to be irradiated RTI may be grasped by the operator visually checking a region in which the melanin concentration is high or may be grasped by using a marker on the object marked on the basis of a past image recorded to the electronic medical record, the treatment plan, and so on.

Light from the light emitting unit 60 desirably includes light in the infrared region, which is a wavelength range in which interaction with a living body is strong. As the index of interaction with a living body, the optical absorption coefficient, the Gruneisen coefficient, or the like can be used. In a case where the optical absorption coefficient is used as the index, it is desirable to select a wavelength so that the optical absorption coefficient of tissue of interest is larger than that of background tissue. The wavelength of the light emitted from the light emitting unit 60 is desirably in a range of 400 nm to 1100 nm, which corresponds to a range from the visible region to the near-infrared region. Specifically the wavelength may be, for example, 532 nm, 1064 nm, 585 nm, 650 nm, or 785 nm at which laser light is easily formed.

The output unit 50 transmits an acoustic wave signal to an acoustic wave device via a wired electric line. The output unit 50 transmits the acoustic wave signal by propagating radio, light, or the like. The acoustic wave signal is transmitted from the output unit 50 to an acoustic wave device including a signal analysis unit via a signal line 70. The signal line 70 may be bundled so as not to obstruct a procedure performed by the operator. The acoustic wave device will be described below.

The connector 30 of this embodiment is configured so as to be fitted together and detachably connected with the target guide 20 (distance guide 20a) of the light irradiation unit 200. The connector 30 is configured so as to connect the light irradiation unit 200 and the probe unit 100 with each other by using elastic force, magnetism, electrostatic force, a difference in atmospheric pressure, friction force, an adhesive capacity, or other actions. In this embodiment, the connector 30 is in the form of a clip; however, a magnetic catch, an electrostatic chuck, a suction cup, a hook and loop fastener, an adhesive material, and so on can be used. A form in which the connector 30 is detachably connected with the handpiece 40 of the light irradiation unit 200 is regarded as a modification to this embodiment. A form in which the connector 30 is configured to be attached to and detached from an extending unit 90 of the probe unit 100 is regarded as a modification to this embodiment.

With the connector 30, the relative positional relationship between the light irradiation unit 200 and the probe unit 100 is fixed as illustrated in FIG. 1C. As a result, as illustrated in FIG. 1E, a relationship in which the irradiation region IR on the object 201 and an acoustically coupling region CR in which the probe 10 is acoustically coupled with the object 201 are mutually stationary is realized.

With the connector 30, as illustrated in FIG. 1D and FIG. 1E, the operator can move the light irradiation unit 200 and the probe unit 100 together toward the region to be irradiated RTI of the object 201. With the connector 30, as illustrated in FIG. 1E, the operator performs a procedure for aligning the irradiation region IR with the region to be irradiated RTI to thereby complete the procedure for aligning the acoustically coupling region CR with the region to be irradiated RTI. In a case where the connector 30 is configured so that the probe unit 100 and the light irradiation unit 200 can be connected with each other with a predetermined intensity, the operator can move the probe unit 100 and the light irradiation unit 200 together toward the object or keep both the probe unit 100 and the light irradiation unit 200 stationary relative to the object 201 by holding only the probe unit 100 or the light irradiation unit 200.

The probe 10 is provided such that the extending unit 90 is interposed between the probe 10 and the connector 30. The extending unit 90 is regarded as a spacer that defines the distance from the connector 30 to the probe 10. The distance between the lower end of the probe 10 and the lower end of the light emitting unit 60 is substantially equal to the distance between the lower end of the target guide 20 and the lower end of the light emitting unit 60.

As illustrated in FIG. 1E, the probe 10 can be acoustically coupled with the object 201 via an acoustic coupling member 211. The probe 10 can be disposed so as to be detachable from the extending unit 90, which allows replacement of the probe 10 due to deterioration or changing of the probe 10 to another type.

The probe 10 of this embodiment is fixed perpendicular to the longitudinal direction (extending direction) of the extending unit 90 but may be connected with the extending unit 90 so as to be inclinable relative to the longitudinal direction. With such a form that allows inclination, the use form in which the handheld light irradiation unit is used in non-perpendicular incidence in which the incidence angle of the irradiation light relative to the object is not 0 degree is allowed. In other words, the probe 10 is rotatably fixed to the extending unit 90 so as to allow the relative angle to the light irradiation unit 200 to be changed.

The probe 10 can be configured such that the inside diameter of the open ring shape is larger than the irradiation region IR (irradiation spot) and is equal to or larger than the outside diameter of the irradiation region guide 20b. The probe 10 and the irradiation region guide 20b of this embodiment are configured so as to be fitted together. As illustrated in FIG. 1C, the connector 30 connects the probe unit 100 with the light irradiation unit 200 so that the open ring portion of the probe 10 and the open ring portion of the irradiation region guide 20b are oriented at the same angle of direction. With such a configuration, the light beam 260 emitted from the light irradiation unit 200 is radiated onto the object 201 without being blocked by the irradiation region guide 20b or the probe 10, and the operator can visually check the region to be irradiated RTI that is irradiated from the light emitting unit 60.

The probe 10 of this embodiment has an open ring shape but may have a closed ring shape. The probe 10 includes at least one transducer that can convert an acoustic wave signal to an electric signal, and may be formed of a probe array in which a plurality of transducers are arranged in a mass or formed of three or more transducers that are discretely disposed.

The probe 10 can be configured so that the reception directional axis of the transducer is oriented to a region in which the light beam 260 diffuses in the object 201 in a state where the light irradiation unit 200 and the probe unit 100 are connected with each other. With such disposition, the effective signal-to-noise (S/N) ratio of the reception signal can be increased. For a similar purpose, a refractive member for producing an acoustic lens effect can be disposed on the object side of the probe 10 so that an acoustic wave from the region in which the light beam 260 diffuses in the object 201 efficiently propagates to the probe 10, The refractive member can be formed as an interface between different members between which the acoustic propagation velocity differs.

The probe unit 100 of this embodiment includes the probe 10 and the connector 30 so that the operator can place the irradiation region IR and the acoustically coupling region CR on the object 201 without a procedure for aligning the region to be irradiated RTI and the irradiation region IR being obstructed.

### Second Embodiment

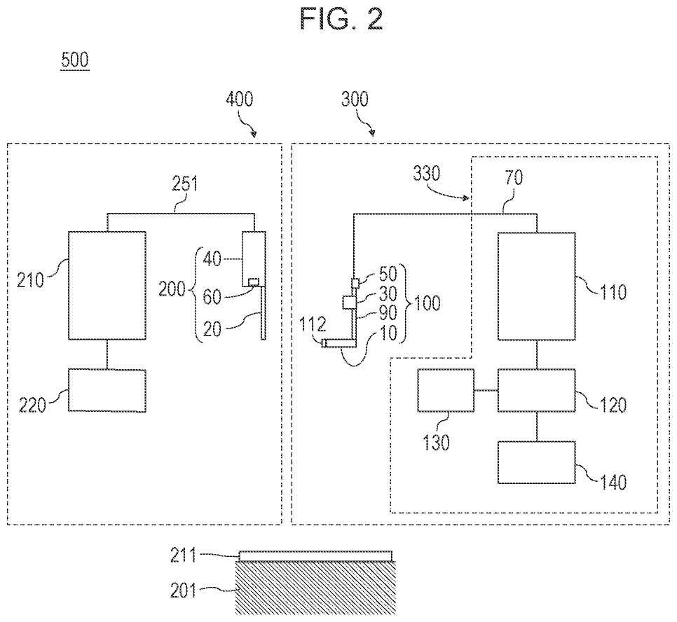

FIG. 2 is a schematic diagram illustrating an irradiation light, monitoring system 500 that includes a light irradiation system 400 and an acoustic wave receiving system 300 according to a second embodiment. For simplification, FIG. 2 illustrates a state where the light irradiation unit 200, which is included in the light irradiation system 400, and the probe unit 100, which is included in the acoustic wave receiving system 300, are not connected with each other. In a case where the light irradiation unit 200 and the probe unit 100 are operated as elements in the irradiation light monitoring system 500, the light irradiation unit 200 and the probe unit 100 are connected with each other as illustrated in FIG. 1C to FIG. 1E.

As illustrated in FIG. 2, the light irradiation system 400 includes a light source 210 that is optically coupled with the light emitting unit 60 via an optical waveguide 251 and a foot switch 220 that controls a timing of the light irradiation from the light source 210 to the light emitting unit 60. The light source 210 is configured so that the wavelength, output, pulse rate, and pulse width of the light source 210 are controlled. The light source 210 and the optical waveguide 251 can be reduced in size so as to be built in the handpiece 40. As the optical waveguide 251, an optical fiber, such as a single-mode fiber, a multimode fiber, or a bundle fiber, or an optical element, such as a mirror, a lens, a diffusing plate, or a quarter-wave plate, can be used.

The foot switch 220 is disposed as a member other than the handpiece 40 from the viewpoint of operability for the operator so as to allow the operator to concentrate on a procedure of holding the handpiece 40. The foot switch 220 can be replaced by another instructing method, such as image recognition for recognizing a gesture made by the operator to identify the instruction or voice recognition for recognizing a voice instruction given by the operator to identify the instruction.

The acoustic wave receiving system 300 includes the probe unit 100 according to the first embodiment and an acoustic wave device 330 that receives an output signal from the output unit 50.

The probe 10 is provided at an object-side end of the extending unit 90 so that the timing at which the light beam 260 is radiated from the light emitting unit 60 and the timing at which the acoustic wave device 330 receives the acoustic wave signal are synchronized with each other via the probe unit 100.

The acoustic wave device 330 includes a signal analysis unit 110 that analyzes a signal sent from the output unit 50, a control unit 120 that controls the signal analysis unit 110 and displays the result of analysis on a monitor 130, the monitor 130, and a console 140 for input of instructions from the operator.

The irradiation light monitoring system 500 analyzes a photoacoustic wave produced in the object 201 by the light beam 260 radiated from the light irradiation system 400 and presents to the operator the degree of effect, on the object 201, of the light beam 260 radiated from the light irradiation system 400.

The degree of effect may be indicated by a numerical value obtained by acoustically monitoring whether a freckle or the like that is a target of treatment is sufficiently decomposed or fragmented or whether the intensity of irradiation of the object is too high to cause a side effect.

Light radiated onto the object 201 reaches an area in the living body not illustrated, which is, for example, hemoglobin in the blood, melanin in the skin, a lesion part, a pigment part, a freckle, a mole, a hair root, or a tattooed part, The area that absorbs the light energy expands and shrinks in volume and produces an acoustic wave (ultrasound wave). The produced photoacoustic wave propagates in the living body, and part thereof reaches the surface of the object 201. Thereafter, the photoacoustic wave is received by the probe unit 100 via the probe 10 acoustically coupled with the object 201.

The photoacoustic wave received by the probe unit 100 passes through the signal line 70 and is transmitted to the signal analysis unit 110 of the acoustic wave device 330 as an electric signal. The signal analysis unit 110 mainly performs a trigger process for obtaining the photoacoustic wave signal and analog/digital conversion. In a case where the photoacoustic wave signal is used as a trigger, the presence or absence of an area that is the source where the photoacoustic wave is produced (hereinafter referred to as “sound source of interest”) can be acoustically detected, but it is not possible to grasp the depth of the sound source of interest in the object 201.

In a case where the probe unit 100 includes a photosensor 112 for triggering, a signal from the photosensor 112 for triggering can be used in the trigger process performed by the signal analysis unit 110. The photosensor 112 for triggering receives light that is radiated from the light irradiation system 400 onto the object 201 and is diffused or reflected on the surface of the object 201, and transmits the received light to the signal analysis unit 110 as an electric signal. The signal analysis unit 110 uses the light radiated onto the surface of the object 201 as a trigger, and therefore, the depth of the sound source of interest in the object 201 can be grasped from the time difference between the timing at which the light is received and the timing at which the photoacoustic wave is received.

In a case where the signal transmitted from the probe unit 100 is a digital signal, the signal analysis unit 110 need not perform analog/digital conversion.

The photoacoustic wave signal subjected to the trigger process and analog/digital conversion in the signal analysis unit 110 is transmitted to the control unit 120. The control unit 120 mainly performs a process for displaying the analyzed photoacoustic wave signal on the monitor 130 and a process for input from the operator via the console 140.

The control unit 120 may display the time-varying waveform of the photoacoustic wave signal processed by the signal analysis unit 110 on the monitor 130. The control unit 120 can display on the monitor 130 whether an area of interest that produces a photoacoustic wave is present on the surface of or in the object 201 on the basis of whether the photoacoustic wave signal exceeds a predetermined threshold. The control unit 120 may display on the monitor 130 the area of interest that produces the photoacoustic wave as a tomographic image. The control unit 120 may display on the monitor 130 the result of detection by the photosensor 112 for triggering.

The operator can specify which data to be displayed on the monitor 130 via the console 140 to the control unit 120. Further, the control unit 120 can output the value of the photoacoustic wave signal and that of the photosensor 112 for triggering to another device (not illustrated) specified by the operator via the console 140.

### Third Embodiment

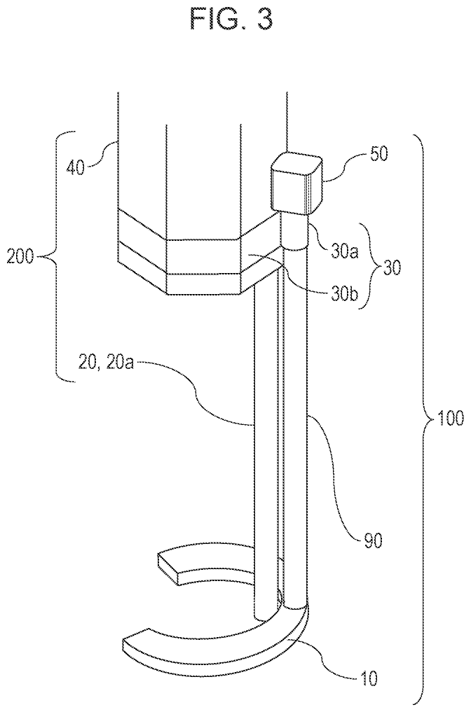

FIG. 3 is a schematic diagram illustrating the probe unit 100 according to a third embodiment, which is a modification to the first embodiment.

The probe unit 100 according to this embodiment is different from the probe unit 100 according to the first embodiment in that the probe unit 100 according to this embodiment is connected with the handpiece 40 of the light irradiation unit 200 by a base part 30a. The connector 30 includes the base part 30a that is fixed to the probe unit 100 and a fastening part 30b that is wrapped around and connected with the handpiece 40.

As the connector 30, a rubber band, a hook and loop fastener, a clamping cap, and so on can be used as in the first embodiment. It is desirable that the connector 30 (30a) is connected with the handpiece 40 at a location so as not to block the light beam radiated from the light emitting unit 60. In other words, the connector 30 in the third embodiment includes the base part 30a and the fastening part 30b.

### Fourth Embodiment

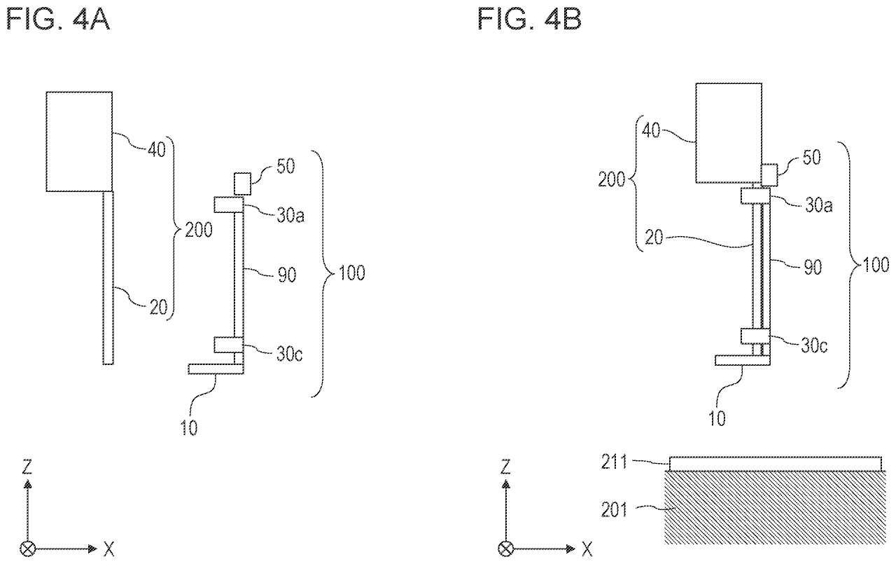

FIG. 4A and FIG. 4B are schematic diagrams illustrating the probe unit 100 according to a fourth embodiment, which is a modification to the first embodiment. FIG. 4A and FIG. 4B respectively illustrate a state where the probe unit 100 and the light irradiation unit 200 are not connected with each other and a state where the probe unit 100 and the light irradiation unit 200 are connected with each other, and correspond to FIG. 1D and FIG. 1E.

The probe unit 100 according to this embodiment is different from the probe unit 100 according to the first embodiment in that the probe unit 100 according to this embodiment is connected with the target guide 20 (distance guide 20a) of the light irradiation unit 200 by a connector 30a and a connector 30c. In this embodiment, the probe unit 100 and the light irradiation unit 200 are connected with each other at a plurality of locations, and therefore, the connection intensity per location is allowed to be low, and it is possible to reduce a risk of one of the units being inclined relative to the irradiation light axis.

A form in which the connector 30a is connected with the handpiece 40 and the connector 30c is connected with the target guide 20 is also regarded as an embodiment of the present disclosure.

### Fifth Embodiment

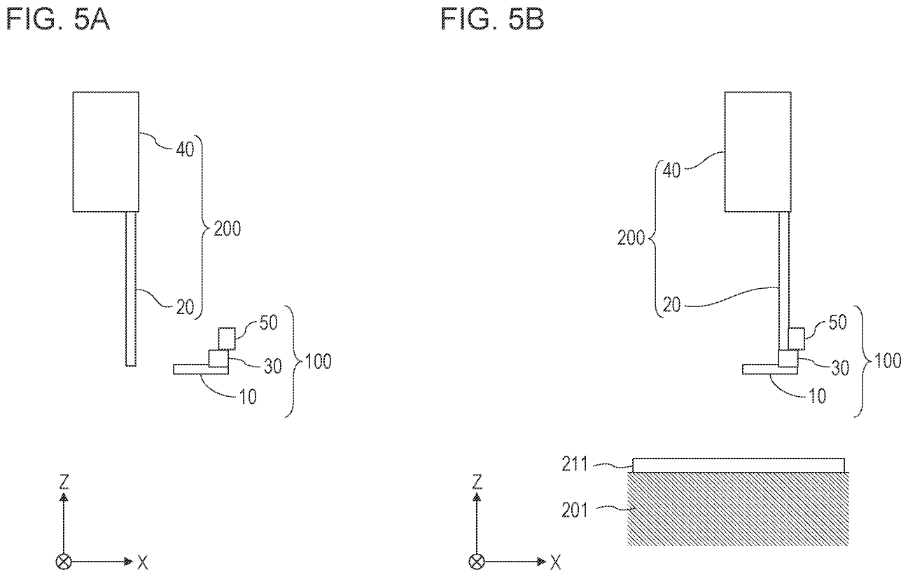

FIG. 5A and FIG. 5B are schematic diagrams illustrating the probe unit 100 according to a fifth embodiment, which is a modification to the first embodiment. FIG. 5A and FIG. 5B respectively illustrate a state where the probe unit 100 and the light irradiation unit 200 are not connected with each other and a state where the probe unit 100 and the light irradiation unit 200 are connected with each other, and correspond to FIG. 1D and FIG. 1E.

As illustrated in FIG. 5A and FIG. 5B, the probe unit 100 of this embodiment is different from the probe unit 100 according to the first embodiment in that the output unit 50 is configured to wirelessly transmit an acoustic wave signal to the acoustic wave device. Further, as illustrated in FIG. 5A and FIG. 5B, the probe unit 100 of this embodiment is different from the probe unit 100 according to the first embodiment in that the extending unit 90 is not included and the connector 30 is configured to be fixed to the distal end side of the target guide 20.

The output unit 50 of this embodiment may include a transceiver not illustrated and a battery that supplies power to the transceiver as units of the probe 10.

### Sixth Embodiment

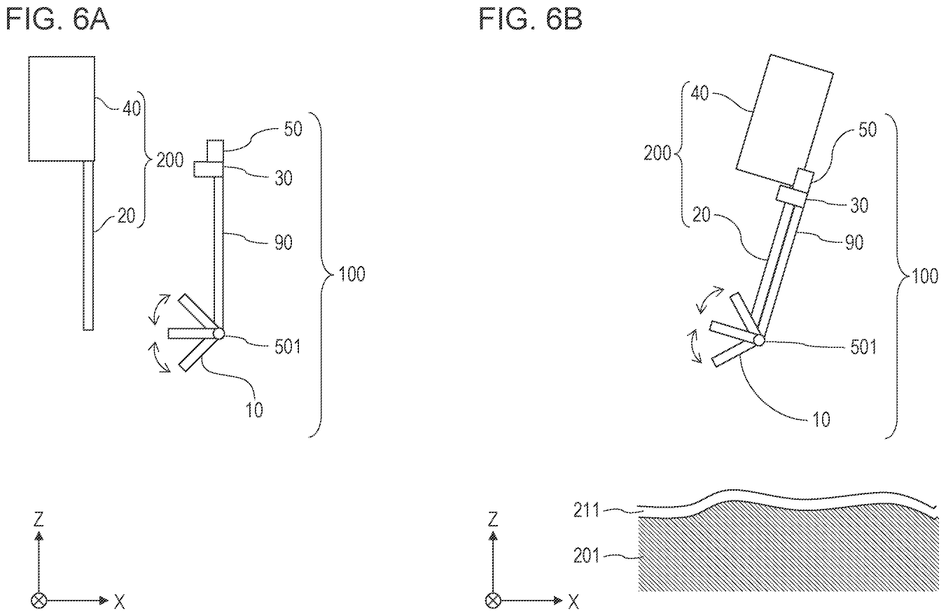

FIG. 6A and FIG. 6B are schematic diagrams illustrating the probe unit 100 according to a sixth embodiment, which is a modification to the first embodiment. FIG. 6A and FIG. 6B respectively illustrate a state where the probe unit 100 and the light irradiation unit 200 are not connected with each other and a state where the probe unit 100 and the light irradiation unit 200 are connected with each other, and correspond to FIG. 1D and FIG. 1E.

The probe 10 of the first embodiment is fixed perpendicular to the longitudinal direction of the extending unit 90. The probe unit 100 of this embodiment is different from the probe unit 100 of the first embodiment in that the probe 10 is connected with the extending unit 90 so as to be inclinable relative to the longitudinal direction of the extending unit 90. When the probe 10 is thus configured so as to be inclinable relative to the extending unit 90, that is, relative to the handpiece 40, limitations on the angle at which the handpiece 40 is held when the light irradiation unit 200 is used are removed. The light irradiation unit 200 is of a handheld type, and therefore, the incidence angle of the irradiation light relative to the object 201 is not 0 degree, and the angle at which the light irradiation unit 200 is held differs from irradiation timing to irradiation timing. Accordingly, the configuration of this embodiment increases usability for the operator.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2018-219742 filed Nov. 22, 2018, which is hereby incorporated by reference herein in its entirely.

What is claimed is:

1. A probe unit for receiving an acoustic wave, the probe unit comprising:
a probe;
an output unit configured to output an acoustic wave signal detected by the probe to an acoustic wave device; and
a connector configured to be detachably connected with a light irradiation unit that includes a light emitting unit and a target guide, wherein
the probe and the connector are disposed so as to allow an operator to visually check a region to be irradiated when the operator places the probe so as to be acoustically coupled with an object.

2. The probe unit according to claim 1, wherein
the probe and the connector are provided at respective positions so as not to overlap with guide light radiated from the light irradiation unit for indicating the region to be irradiated.

3. The probe unit according to claim 1, wherein
the probe and the connector are provided at respective positions so as not to overlap with a light beam from the light emitting unit.

4. The probe unit according to claim 1, wherein
the probe and the connector are provided at respective positions so as to surround a light beam from the light emitting unit.

5. The probe unit according to claim 1, wherein
the probe has a dosed ring shape or an open ring shape and includes three or more discretely disposed transducers.

6. The probe unit according to claim 1, wherein
the connector is configured to be connected with the target guide.

7. The probe unit according to claim 1, further comprising
an extending unit configured to be connected with the probe and with the connector.

8. The probe unit according to claim 1, wherein
the probe is provided so as to allow an angle of the probe relative to the light irradiation unit to be changed.

9. The probe unit according to claim 8, wherein
the probe is rotatably fixed to the extending unit so as to allow the angle of the probe relative to the light irradiation unit to be changed.

10. The probe unit according to claim 8, wherein
the light irradiation unit further includes a handpiece on which the light emitting unit is provided and that is configured to be held by the operator, and
the probe is rotatably fixed to the extending unit so as to allow an angle of the probe relative to the handpiece to be changed.

11. The probe unit according to claim 1, wherein
the output unit is configured to transmit the acoustic wave signal to the acoustic wave device wirelessly, in a wired manner, or by light.

12. The probe unit according to claim 1, further comprising
a photosensor arranged to detect light from the light emitting unit.

13. The probe unit according to claim 12, wherein
the photosensor is disposed at the probe.

14. An acoustic wave receiving system comprising:
the probe unit according to claim 1; and
an acoustic wave device configured to receive an acoustic wave signal from the probe unit.
